SEQUENCE LISTING (43 sequences)

SEQ ID NO: 1 - RNA, 22 nt, Human
ugagguaguagguuguauaguu

SEQ ID NO: 2 - RNA, 22 nt, Human
ugagguaguagguugugugguu

SEQ ID NO: 3 - RNA, 22 nt, Human
ugagguaguagguuguaugguu

SEQ ID NO: 4 - RNA, 21 nt, Human
agagguaguagguuccauagu

SEQ ID NO: 5 - RNA, 21 nt, Human
ugagguaggagguuguauagu

SEQ ID NO: 6 - RNA, 22 nt, Human
ugagguaguagauuguauaguu

SEQ ID NO: 7 - RNA, 22 nt, Human
uagcagcacauaaugguuugug

SEQ ID NO: 8 - RNA, 22 nt, Human
uagcagcacguaaauauuggcg

SEQ ID NO: 9 - RNA, 20 nt, Human
acugcagugaaggcacuugu

SEQ ID NO: 10 - RNA, 22 nt, Human
uaaggugcaucuagugcagaua

SEQ ID NO: 11 - RNA, 23 nt, Human
ugugcaaaucuaugcaaaacuga

SEQ ID NO: 12 - RNA, 23 nt, Human
ugugcaaauccaugcaaaacuga

SEQ ID NO: 13 - RNA, 23 nt, Human
ugugcaaauccaugcaaaacuga

SEQ ID NO: 14 - RNA, 22 nt, Human
uagcuuaucagacugauguuga

SEQ ID NO: 15 - RNA, 22 nt, Human
aagcugccaguugaagaacugu

SEQ ID NO: 16 - RNA, 21 nt, Human
aucacauugccagggauuucc

SEQ ID NO: 17 - RNA, 22 nt, Human
uggcucaguucagcaggaacag

SEQ ID NO: 18 - RNA, 22 nt, Human
cauugcacuugucucggucuga

SEQ ID NO: 19 - RNA, 22 nt, Human
uucaaguaauccaggauaggcu

SEQ ID NO: 20 - RNA, 22 nt, Human
uucaaguaauucaggauagguu

SEQ ID NO: 21 - RNA, 22 nt, Human
uucacaguggcuaaguuccgcu

SEQ ID NO: 22 - RNA, 22 nt, Human
aaggagcucacagucuauugag

SEQ ID NO: 23 - RNA, 22 nt, Human
cuagcaccaucugaaaucgguu

SEQ ID NO: 24 - RNA, 22 nt, Human
cuuucagucggauguuugcagc

SEQ ID NO: 25 - RNA, 21 nt, Human
ggcaagaugcuggcauagcug

SEQ ID NO: 26 - RNA, 21 nt, Human
uauugcacauuacuaaguugc

SEQ ID NO: 27 - RNA, 19 nt, Human
gugcauuguaguugcauug

SEQ ID NO: 28 - RNA, 22 nt, Human
uggaauguaaagaaguauggag

SEQ ID NO: 29 - RNA, 23 nt, Human
uggaagacuagugauuuuguugu

SEQ ID NO: 30 - RNA, 23 nt, Human
ucuuugguuaucuagcuguauga

SEQ ID NO: 31 - RNA, 21 nt, Human
acccuguagauccgaauuugu

SEQ ID NO: 32 - DNA, 13 bp, Artificial
  artificially generated let-7a probe
aactatacaacct

SEQ ID NO: 33 - DNA, 11 bp, Artificial
  artificially generated miR-30 probe
gctgcaaacat

SEQ ID NO: 34 - DNA, 12 bp, Artificial
  artificially generated miR-20 probe
tacctgcactat

SEQ ID NO: 35 - DNA, 12 bp, Artificial
  artificially generated miR-16 probe
tacctgcactat

SEQ ID NO: 36 - DNA, 12 bp, Artificial
  artificially generated let-7a probe
tacctgcactat

SEQ ID NO: 37 - DNA, 11 bp, Artificial
  artificially generated miR-30 probe
ccgactgaaag

SEQ ID NO: 38 - DNA, 10 bp, Artificial
  artificially generated miR-20 probe
aagcacttta

SEQ ID NO: 39 - DNA, 10 bp, Artificial
  artificially generated miR-16 probe
aagcacttta

SEQ ID NO: 40 - DNA, 50 bp, Artificial
  artificially generated miR-20 target probe
ttgcctgctcgacttagattttttgcactgccaagacttacctgcactat

SEQ ID NO: 41 - DNA, 30 bp, Artificial
  artificially generated miR-20 target probe
aaggactttaatcactggatagcgacgtgg

SEQ ID NO: 42 - DNA, 18 bp, Artificial
  artificially generated PCR primer
ttgcctgctcgacttaga

SEQ ID NO: 43 - DNA, 15 bp, Artificial
  artificially generated PCR primer
tgcactgccaagact

(19) United States
(12) Patent Application Publication
(10) Pub. No.: US 2006/0003337 A1
(43) Pub. Date: Jan. 5, 2006

(54) Detection of small RNAS

(76) Inventors: John Brandis, Hercules, CA (US); Elena V. Bolchakova, Union City, CA (US); Achim E. Karger, Foster City, CA (US)

Correspondence Address: HARNESS, DICKEY & PIERCE, P.L.C., P.O. BOX 828, BLOOMFIELD HILLS, MI, 48303, US

(21) Appl. No.: 10/881,362

(22) Filed: Jun. 30, 2004

(51) Int. Cl. C12Q 1/68; C12P 19/34

(52) U.S. Cl. 435/6; 435/91.2

(57) ABSTRACT

Methods for detecting an RNA such as a small RNA comprising up to about 40 nucleotides are disclosed. The methods comprise forming a ligation mixture comprising a sample suspected of comprising the small RNA, a ligase and a target probe set. The target probe set comprises a first target probe comprising a 3′ portion that hybridizes to the small RNA and a 5′ portion having a first PCR primer target sequence, and a second target probe comprising a 5′ portion that hybridizes to the RNA immediately adjacent to the 3′ end of the first target probe and a 3′ portion having a second PCR primer target sequence. The target probe set hybridizes to the RNA and ligates to form a probe set ligation sequence. A probe set ligation sequence can be amplified and detected using a polymerase chain reaction.

Detection of RNA molecules is fundamental to molecular biology, in research, industrial, agricultural and clinical settings. Among the types of RNA that are of interest are naturally occurring and synthetic regulatory RNAs such as small RNA molecules (Lee, et al., Science 294: 862-864, 2001; Ruvkun, Science 294: 797-799; Pfeffer et al., 304: Science 734-736, 2004; Ambros, Cell 107: 823-826, 2001; Ambros et al., RNA 9: 277-279, 2003; Carrington and Ambros, Science 301: 336-338, 2003; Reinhart et al., Genes Dev. 16: 1616-1626, 2002 Aravin et al., Dev. Cell 5: 337-350, 2003, Tuschel et al., Science 294: 853-858, 2001; Susi P. et al., Plant Mol. Biol. 54: 157-174, 2004; Xie et al., PLoS Biol. 2: E104, 2004). Small RNA molecules, such as, for example, micro RNAs (mRNA), short interfering RNAs (siRNA), small temporal RNAs (stRNA) and short nuclear RNAs (snRNA), can be, typically, less than about 40 nucleotides in length and can be of low abundance in a cell. Because of their small size and low abundance, detection and quantification of small RNAs can be difficult or unreliable using RNA detection methods such as reverse transcription-polymerase chain reaction assays, RNase protection assays or Northern blot assays. Such assays can be lacking in sensitivity or can be quantitatively or qualitatively inaccurate.

### SUMMARY

Accordingly, the inventors herein have succeeded in devising a new approach to detecting RNA in a sample. The approach is based upon RNA-templated ligation and polymerase chain reaction (PCR) amplification.

The present invention provides methods for determining the presence of an RNA in a sample. The RNA can be a naturally occurring RNA such as a small RNA, or a synthetic RNA. A small RNA can be, for example, an mRNA, an siRNA, an stRNA or an snRNA.

In various embodiments, the present invention can involve methods for detecting a small RNA. The methods can comprise forming a ligation mixture comprising a sample suspected of comprising the small RNA, a ligase and a target probe set for detecting the small RNA. The target probe set can comprise a first target probe comprising a 3′ portion that hybridizes to the small RNA and a 5′ portion having a first PCR primer target sequence, and a second target probe comprising a 5′ portion that hybridizes to the RNA immediately adjacent to the 3′ end of the first target probe and a 3′ portion having a second PCR primer target sequence, wherein the mixture is formed under conditions in which a target probe set hybridizes to the small RNA and ligates to form a probe set ligation sequence. The methods can further comprise forming a detection mixture comprising a probe set ligation sequence, a first PCR primer which hybridizes to the complement of the first PCR primer target sequence and a second PCR primer which hybridizes to the second PCR primer target sequence. Any probe set ligation sequence comprised by the detection mixture can be amplified using a polymerase chain reaction. Detection of amplification of any probe ligation sequence can be indicative of a small RNA comprised by the sample.

In various embodiments, the methods of the present invention can involve detecting an RNA by forming a mixture comprising a sample suspected of comprising a small RNA, a target probe set comprising a first target probe and a second target probe. The first target probe can comprise a 3′ portion that hybridizes to the small RNA and a 5′ portion having a first PCR primer target sequence. The second target probe can comprise a 5′ portion that hybridizes to the small RNA immediately adjacent to the 3′ end of the first target probe and a 3′ portion having a second PCR primer target sequence. At least one of the first or second target probes further comprises an affinity tag. In addition, the mixture can contain an affinity tag binding partner, which can be covalently attached to a solid phase support. Upon formation of the mixture, a complex can form comprising a solid phase support, the affinity tag binding partner, the target probe comprising the affinity tag, the small RNA, and a target probe which hybridizes to the small RNA immediately adjacent to the target probe comprising the affinity tag. In some configurations, the mixture can be washed, thereby removing first target probe and second target probe which is not hybridized to the small RNA. A ligase such as a T4 ligase can be added to a mixture comprising the complex bound to the solid phase support to form a probe set ligation sequence.

In various configurations, a detection mixture can be formed comprising a probe set ligation sequence, a first PCR primer that hybridizes to the complement of the first PCR primer target, a second PCR primer that hybridizes to the second PCR primer target, and a DNA polymerase. A probe set ligation sequence comprised by the mixture can be amplified using a polymerase chain reaction. Detection of amplification of a probe ligation sequence can be indicative of a small RNA comprised by the sample.

In various embodiments, the present invention can include methods for detecting a plurality of small RNAs in a sample. The methods can comprise forming a ligation mixture that comprises (a) a sample suspected of comprising the plurality of small RNAs; (b) a ligase; and (c) a plurality of target probe sets, each target probe set comprising (i) a first target probe comprising 5′ portion having a first PCR primer target sequence and a 3′ portion that hybridizes to a small RNA and (ii) a second target probe comprising a 5′ portion that hybridizes to the small RNA immediately adjacent to the 3′ end of the first target probe and a 3′ portion having a second PCR primer target sequence. The ligation mixture can be formed under conditions in which each target probe set hybridizes to a target small RNA to which it is complementary, and ligates to form a plurality of probe set ligation sequences. A detection mixture can be formed comprising the plurality of probe set ligation sequences, a first PCR primer which hybridizes to the complement of the first PCR primer targets, a second PCR primer which hybridizes to the second PCR primer targets, and a DNA polymerase. Each probe set ligation sequence can be amplified using a polymerase chain reaction. Detection of amplification of any probe ligation sequence can be indicative of a small RNA comprised by the sample.

The invention also provides, in various embodiments, methods of detecting a plurality of small RNAs. The methods can comprise forming a hybridization mixture comprising (a) a sample suspected of comprising a plurality of small RNAs, (b) a plurality of target probe sets for detecting the small RNA, each target probe set comprising (i) a first target probe comprising a 3′ portion that hybridizes to a small RNA and a 5′ portion comprising a first PCR primer target sequence, and (ii) a second target probe comprising a 5′ portion that hybridizes to a small RNA immediately adjacent to the 3′ end of the first target probe and a 3′ portion having a second PCR primer target sequence, in which at least one of the first target probe and the second target probe further comprises an affinity tag, and (c) a binding partner for the affinity tag bound to a solid phase support. The mixture is formed under conditions in which a target probe set hybridizes to a small RNA. The method further involve washing the hybridization mixture, thus formed, to remove first target probes and second target probes which are not hybridized to a small RNA. The method can further involve contacting the hybridization mixture with a ligase to form a plurality of probe set ligation sequences. A detection mixture is then formed. The detection mixture can comprise a plurality of probe set ligation sequences, a first PCR primer which hybridizes to the complement of the first PCR primer target, a second PCR primer which hybridizes to the second PCR primer target, and a DNA polymerase. Any probe set ligation sequence present in the detection mixture is then amplified using a polymerase chain reaction and detected.

In various embodiments, the present invention can comprise a mixture for detection of a small RNA. The mixture can comprise a target probe set for detecting a small RNA comprising from about 10 to about 40 nucleotides. The target probe set can comprise a first target probe comprising a 3′ portion that hybridizes to a small RNA and a 5′ portion having a first PCR primer target sequence, and a second target probe comprising a 5′ portion that hybridizes to the small RNA immediately adjacent to the 3′ end of the first target probe and a 3′ portion having a second PCR primer target sequence. The target probe can further comprise a detection probe hybridization sequence, as well as comprise an affinity tag. In some configurations, the mixture can further comprise a sample suspected of comprising a small RNA that comprises from about 10 to about 40 nucleotides, and a ligase.

In various embodiments, the present invention can comprise a detection mixture for detecting a small RNA comprising from about 10 to about 40 nucleotides. The detection mixture can comprise (a) a probe set ligation sequence comprising a first PCR primer target site, a second PCR primer target site, and a sequence complementary to a small RNA comprising from about 10 to about 40 nucleotides, (b) a first PCR primer which hybridizes to the complement of the first PCR primer target sequence and (c) a second PCR primer which hybridizes to the second PCR primer target sequence. In various configurations, the probe set ligation sequence can further comprise a detection probe hybridization sequence, and in certain aspects, a probe set ligation sequence can further comprise an affinity tag.

The detection mixture can further comprise a DNA polymerase. The DNA polymerase can be a thermostable DNA polymerase, such as a taq polymerase. In certain aspects, the DNA polymerase can be a DNA polymerase having 5′ nuclease activity.

In various other embodiments, the present invention can include a kit for the detection of small RNA. The kit can comprise a first target probe comprising a 3′ portion that hybridizes to a small RNA and a 5′ portion having a first PCR primer target sequence and a second target probe comprising a 5′ portion that hybridizes to the small RNA immediately adjacent to the 3′ portion of the first target probe and a 3′ portion having a second PCR primer target sequence, wherein upon hybridization of the first and second target probes to the small RNA in the presence of a ligase, a ligation sequence can be formed. Components of the kit can be packaged in a container.

In various embodiments, the RNA detected by methods of the present invention can be at least ten nucleotides in length. The RNA detected by methods of the present invention can be, for example, from about 10 nucleotides to about 500 nucleotides in length, from above 12 nucleotides to about 250 nucleotides in length, from about 15 nucleotides to about 100 nucleotides in length, from about 18 contiguous nucleotides to about 40 nucleotides in length, from 20 to about 40 contiguous nucleotides in length, or from about 20 to about 30 contiguous nucleotides in length. The RNA detected by methods of the present invention can consist of, for example, 20, 21, 22, 23, 24, 25, 26, 27, 28, 29, or 30 contiguous nucleotides. The RNA detected by these methods can be a naturally occurring RNA, or, in some configurations, a synthetic RNA. The small RNA detected using the disclosed methods can be, in non-limiting example, an mRNA, an stRNA, an siRNA, or an snRNA. Some non-limiting examples of species of small RNA that can be detected include let-7a, miR-16, miR-20, and miR-30.

In certain configurations, a 3′ portion of the a target probe that hybridizes to a small RNA and a 5′ portion of the second target probe that hybridizes to the small RNA together can have a total of not more than 40 nucleotides, or in some configurations, a total of not more than 25 nucleotides.

In various embodiments of the invention, at least one target probe can further comprise a detection probe hybridization sequence. In some configurations, the detection mixture can further comprise a detection probe. The detection probe can comprise a nucleic acid sequence that hybridizes to the detection probe hybridization sequence.

In various embodiments, a detection methods can utilize any probe which can detect a nucleic acid sequence. In some embodiments, a detection method can comprise a fluorescence assay in which a fluorescence signal can be detected that can be indicative of probe binding to its target. In some configurations, a detection probe can be, for example, a fluorogenic 5′-exonuclease assay probe such as a TaqMan® probe described herein, various stem-loop molecular beacons, stemless or linear beacons, PNA Molecular Beacons™, linear PNA beacons, non-FRET probes, Sunrise®/Amplifluor® probes, stem-loop and duplex Scorpion™ probes, bulge loop probes, pseudo knot probes, cyclicons, MGB Eclipse™ probe (Epoch Biosciences), hairpin probes, peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Labeling probes can also comprise black hole quenchers (Biosearch), Iowa Black (IDT), QSY quencher (Molecular Probes), and Dabsyl and Dabcel sulfonate/carboxylate Quenchers (Epoch). Labeling probes can also comprise sulfonate derivatives of fluorescent dyes, phosphoramidite forms of fluorescein, or phosphoramidite forms of CY5. In some embodiments, interchelating labels can be used such as ethidium bromide, SYBR® Green I, and PicoGreen®, thereby allowing visualization in real-time, or end point, of an amplification product in the absence of a labeling probe.

In various configurations, a detection probe can comprise a fluorophore and a fluorescence quencher. The detection probe, in these embodiments, can be used in a 5′ nuclease assay such as a fluorogenic 5′ nuclease assay, such as a Taqman® assay, in which the fluorophore or the fluorescence quencher is released from the detection probe if the detection probe is hybridized to the detection probe hybridization sequence. In these embodiments, the 5′ nuclease assay can utilize 5′ nucleolytic activity of a DNA polymerase that catalyzes a PCR amplification of a probe set ligation sequence. The fluorogenic 5′ nuclease detection assay can be a real-time PCR assay or an end-point PCR assay. The fluorophore comprised by a detection probe in these embodiments can be any fluorophore that can be tagged to a nucleic acid, such as, for example, FAM, VIC, Sybra Green, TET, HEX, JOE, NED, LIZ, TAMRA, ROX, ALEXA, Texas Red, Cy3, Cy5, Cy7, Cy9, or dR6G.

In some configurations, at least one of a first PCR primer and a second PCR primer can further comprise a fluorophore. In these configurations, detecting the amplified probe set ligation sequence can comprise electrophoretically separating the detection mixture, for example in a medium which separates molecules according to intrinsic charge and frictional coefficient, and stimulating fluorescence of a fluorophore comprised by irradiation with an excitation wavelength for a fluorophore.

In some configurations, amplification of a probe set ligation sequence can be detected by hybridizing a detection probe to a probe set ligation sequence. Hybridization of the detection probe can be detected using any analytical method known in the art, for example electrophoresis through a medium and irradiation to excite a fluorophore comprised by a detection probe.

In embodiments of the invention which utilize an affinity tag, the affinity tag can be a ligand for a binding partner, such as a hapten for an antibody. An affinity tag can be, in non-limiting example, biotin or digoxygenin, and an affinity tag binding partner can be, for example, streptavidin, avidin, an antibody directed against biotin, or an antibody directed against digoxygenin. In some embodiments, an affinity tag binding partner can be covalently bound to a solid phase support. A solid phase support can be, for example, a solid matrix such as, for example, paramagnetic beads. In some configurations, the amplified probe set comprising a detection probe hybridization sequence and an affinity tag, and which is immobilized on a solid phase support comprising an affinity tag binding partner, can be probed with a detection probe that hybridizes to the detection probe hybridization sequence. In some configurations of these embodiments, the detection probe can comprise a fluorophore. The detection probe in these configurations can be detected using a fluorogenic assay such as an assay described above, or can be detected according to its mobility in an electrophoresis medium such as, for example, a capillary gel.

In some embodiments, the mixture can comprise a target probe comprising an affinity tag. The mixture can also comprise a binding partner for the affinity tag. The binding partner can be bound to a solid phase support. In these embodiments, a complex can form so that the mixture can be washed to remove target probe that is not hybridized to the small RNA. A ligating agent can be added to the mixture comprising the complex bound to the solid phase support to form a probe set ligation sequence, which can be amplified using a polymerase chain reaction. Methods of these embodiments can detect as few as 60,000 copies of a small RNA in a sample, or as few as 120,000 copies of a small RNA in a sample. Methods of these embodiments can detect less than 1 attomole of a small RNA.

Ligating according to the present teachings can comprise any enzymatic or non-enzymatic method wherein an inter-nucleotide linkage is formed between apposed ends of an upstream probe and a downstream probe that are adjacently hybridized to a template. Apposed ends of the annealed nucleic acid probes can be suitable for ligation. In some embodiments, ligation can also comprise at least one gap-filling procedure, wherein the ends of the two probes are not adjacently hybridized initially but the 3′-end of the upstream probe can be extended by one or more nucleotides until it is adjacent to the 5′-end of the downstream probe, for example, by a filling-in reaction using a polymerase (see, e.g., U.S. Pat. No. 6,004,826). The internucleotide linkage can include, but is not limited to, phosphodiester bond formation. Such bond formation can include, without limitation, a bond formed using a ligating agent. A ligating agent can include an enzymatic agent such as a DNA ligase or an RNA ligase, such as, for example, T4 DNA ligase, T4 RNA ligase, Thermus thermophilus (Tth) ligase, Thermus aquaticus (Taq) DNA ligase, Thermus scotoductus (Tsc) ligase, TS2126 RNA ligase, Archaeoglobus flugidus (Afu) ligase, or Pyrococcus furiosus (Pfu) ligase. In some configurations, a ligase can be a reversibly inactivated ligase such as disclosed in U.S. Pat. No. 5,773,258, as well as enzymatically active mutants and variants thereof.

Ligating, in some embodiments, can also comprise forming of other internucleotide linkages. These can include, in some configurations, forming a covalent linkage such as a thiophosphorylacetylamino linkage. In certain configurations, other covalent linkages can be formed such as a 5′-phosphorothioester, or a pyrophosphate linkage. Various chemical ligating agents known to skilled artisans can be used to establish such linkages.

Ligating can, in certain embodiments, occur spontaneously such as by autoligation. In certain configurations, activating or reducing agents can be used.

In embodiments involving methods of detecting a plurality of small RNAs in a sample, amplifying a probe set ligation sequence can yield an amplification product that has an electrophoretic mobility which differs from that of other probe set ligation sequences of the sample.

### BRIEF DESCRIPTION OF THE DRAWINGS

FIG. 1 illustrates a schematic of a method for micro RNA detection using RNA-templated ligation and PCR amplification.

FIG. 2 illustrates a time course of RNA-templated deoxyribooligonucleotide ligation using T4 ligase and four different synthetic micro RNA templates.

FIG. 3 illustrates the effect of nick position on ligation rate of GAPDH RNA-templated deoxyribooligonucleotide ligation using T4 ligase.

FIG. 4 illustrates the effect of nick position on ligation rate of let-7a micro RNA-templated deoxyribooligonucleotide ligation using T4 ligase.

FIG. 5 illustrates detection of micro RNA miR-20 by ligation and a fluorogenic 5′ nuclease real-time PCR amplification assay.

FIG. 6 illustrates a histogram plot of Ct values in detection of synthetic micro RNA miR-20 using RNA-templated ligation and a fluorogenic 5′ nuclease real-time PCR amplification assay.

FIG. 7 illustrates a histogram plot of Ct values in an experimental investigation of sensitivity of detection of micro RNAs miR-20 and let-7a comprised by HeLa cells using RNA-templated ligation and a fluorogenic 5′ nuclease assay PCR amplification.

FIG. 8 illustrates a schematic of a method for RNA detection comprising formation of a complex bound to a solid support, washing the complex, ligating deoxyribonucleotides bound to the complex, and amplifying the ligation product.

FIG. 9 illustrates experimental investigation of additives for enhancement of ligation reaction rates.

### DETAILED DESCRIPTION

Methods and compositions for detecting RNA in a sample are described herein. The methods and compositions described herein utilize laboratory techniques well known to skilled artisans and can be found in laboratory manuals such as Sambrook, J., et al., Molecular Cloning: A Laboratory Manual, 3^{rd} ed. Cold Spring Harbor Laboratory Press, Cold Spring Harbor, N.Y., 2001; D. L. et al., Cells: A Laboratory Manual, Cold Spring Harbor Laboratory Press, Cold Spring Harbor, N.Y., 1998; and Harlow, E., Using Antibodies: A Laboratory Manual, Cold Spring Harbor Laboratory Press, Cold Spring Harbor, N.Y., 1999.

The present invention provides, in various embodiments, methods for determining the presence in a sample of an RNA. The RNA can be a naturally occurring RNA or a synthetic RNA such as a small RNA. A small RNA can be, for example, a micro RNA (mRNA), a short interfering RNA (siRNA), a short temporal RNA (stRNA) or a small nuclear RNA (snRNA). A microRNA detected by the disclosed methods can be, for example, a microRNA described in a database such as a searchable database on the internet, for example “the mRNA Registry” which is accessible on the World Wide Web at http://www.sanger.ac.uk/Software/Rfam/mirna/index.shtml. An RNA detected by the disclosed methods can also be a synthetic nucleobase polymer such as, for example, a nucleobase polymer comprising a thiophosphate group in place of a phosphate group. Determining the presence of an RNA can comprise determining the presence, absence or quantity of the RNA.

In various embodiments, an RNA detected by methods of the present invention can be at least about 10 nucleotides in length. An RNA detected by methods of the present invention can be, for example, from about 10 nucleotides to about 500 nucleotides in length, from about 12 nucleotides to about 250 nucleotides in length, from about 15 nucleotides to about 100 nucleotides in length, from about 18 nucleotides to about 40 nucleotides in length, or from about 20 to about 30 nucleotides in length. An RNA detected by methods of the present invention can consist of, for example, 20, 21, 22, 23, 24, 25, 26, 27, 28, 29, or 30 contiguous nucleotides. An RNA detected by these methods can be a naturally occurring RNA, or, in some configurations, a synthetic RNA. A small RNA detected using the disclosed methods can be, in non-limiting example, an mRNA, an stRNA, an siRNA, or an snRNA. Some non-limiting examples of species of small RNA that can be detected include let-7a, miR-16, miR-20, and miR-30. Some non-limiting examples of human mRNAs are listed in Table 1.

TABLE 1

| | | Identi- |
|---|---|---|
| Name of miRNA | Sequence (5′ to 3′) | fication |
| let-7a | UGAGGUAGUAGGUUGUAUAGUU | SEQ ID NO: 1 |
| let-7b | UGAGGUAGUAGGUUGUGUGGUU | SEQ ID NO: 2 |
| let-7c | UGAGGUAGUAGGUUGUAUGGUU | SEQ ID NO: 3 |
| let-7d | AGAGGUAGUAGGUUCCAUAGU | SEQ ID NO: 4 |
| let-7e | UGAGGUAGGAGGUUGUAUAGU | SEQ ID NO: 5 |
| let-7f | UGAGGUAGUAGAUUGUAUAGUU | SEQ ID NO: 6 |
| miR-15 | UAGCAGCACAUAAUGGUUUGUG | SEQ ID NO: 7 |
| miR-16 | UAGCAGCACGUAAAUAUUGGCG | SEQ ID NO: 8 |
| miR-17 | ACUGCAGUGAAGGCACUUGU | SEQ ID NO: 9 |
| miR-18 | UAAGGUGCAUCUAGUGCAGAUA | SEQ ID NO: 10 |
| miR-19a* | UGUGCAAAUCUAUGCAAAACUGA | SEQ ID NO: 11 |
| miR-19b* | UGUGCAAAUCCAUGCAAAACUGA | SEQ ID NO: 12 |
| miR-20 | UAAAGUGCUUAUAGUGCAGGUA | SEQ ID NO: 13 |
| miR-21 | UAGCUUAUCAGACUGAUGUUGA | SEQ ID NO: 14 |
| miR-22 | AAGCUGCCAGUUGAAGAACUGU | SEQ ID NO: 15 |
| miR-23 | AUCACAUUGCCAGGGAUUUCC | SEQ ID NO: 16 |
| miR-24 | UGGCUCAGUUCAGCAGGAACAG | SEQ ID NO: 17 |
| miR-25 | CAUUGCACUUGUCUCGGUCUGA | SEQ ID NO: 18 |
| miR-26a* | UUCAAGUAAUCCAGGAUAGGCU | SEQ ID NO: 19 |
| miR-26b* | UUCAAGUAAUUCAGGAUAGGUU | SEQ ID NO: 20 |
| miR-27 | UUCACAGUGGCUAAGUUCCGCU | SEQ ID NO: 21 |
| miR-28 | AAGGAGCUCACAGUCUAUUGAG | SEQ ID NO: 22 |
| miR-29 | CUAGCACCAUCUGAAAUCGGUU | SEQ ID NO: 23 |
| miR-30 | CUUUCAGUCGGAUGUUUGCAGC | SEQ ID NO: 24 |
| miR-31 | GGCAAGAUGCUGGCAUAGCUG | SEQ ID NO: 25 |
| miR-32 | UAUUGCACAUUACUAAGUUGC | SEQ ID NO: 26 |
| miR-33 | GUGCAUUGUAGUUGCAUUG | SEQ ID NO: 27 |
| miR-1 | UGGAAUGUAAAGAAGUAUGGAG | SEQ ID NO: 28 |
| miR-7 | UGGAAGACUAGUGAUUUUGUUGU | SEQ ID NO: 29 |
| miR-9 | UCUUUGGUUAUCUAGCUGUAUGA | SEQ ID NO: 30 |
| miR-10 | ACCCUGUAGAUCCGAAUUUGU | SEQ ID NO: 31 |

In various embodiments, methods for detecting a small RNA can include forming a ligation mixture comprising a sample suspected of comprising the small RNA, a ligating agent such as a ligase and a target probe set for detecting the small RNA. A sample suspected of comprising an RNA can be a sample believed to comprise the RNA. The sample can be, for example, a diagnostic sample such as blood or a biopsy sample, or an investigational sample such as, for example, a tissue sample from a plant or animal, or a sample from a culture of a microorganism such as a eukaryotic microorganism, for example a yeast.

Ligating according to the present teachings can comprise any enzymatic or non-enzymatic method wherein an inter-nucleotide linkage is formed between apposed ends of an upstream probe and a downstream probe that are adjacently hybridized to a template. Apposed ends of the annealed nucleic acid probes can be suitable for ligation. In some embodiments, ligation can also comprise at least one gap-filling procedure, wherein the ends of the two probes are not adjacently hybridized initially but the 3′-end of the upstream probe can be extended by one or more nucleotides until it is adjacent to the 5′-end of the downstream probe, for example, by a filling-in reaction using a polymerase (see, e.g., U.S. Pat. No. 6,004,826). The internucleotide linkage can include, but is not limited to, phosphodiester bond formation. Such bond formation can include, without limitation, a bond formed using a ligating agent. A ligating agent can include an enzymatic agent such as a DNA ligase or an RNA ligase, such as, for example, T4 DNA ligase, T4 RNA ligase, Thermus thermophilus (Tth) ligase, Thermus aquaticus (Taq) DNA ligase, Thermus scotoductus (Tsc) ligase, TS2126 RNA ligase, Archaeoglobus flugidus (Afu) ligase, or Pyrococcus furiosus (Pfu) ligase. In some configurations, a ligase can be a reversibly inactivated ligase such as disclosed in U.S. Pat. No. 5,773,258, as well as enzymatically active mutants and variants thereof.

Ligating, in some embodiments, can also comprise forming of other internucleotide linkages. These can include, in some configurations, a covalent linkage such as a thiophosphorylacetylamino linkage. This type of bond can be formed between reactive groups such as an α-haloacyl group and a phosphothioate group. In certain configurations, other ligating agents can comprise a phosphorothioate, a tosylate or iodide group which can form a 5′-phosphorothioester, or a pyrophosphate linkage.

Ligating can, in certain embodiments, occur spontaneously such as by autoligation. In certain configurations, activating or reducing agents can be used. Examples of activating and reducing agents can include, without limitation, carbodiimide, cyanogen bromide (BrCN), imidazole, 1-methylimidazole/carbodiimide/cystamine, N-cyanoimidazole, dithiothreitol (DTT) and ultraviolet light, such as used for photoligation using routine methods known to skilled artisans. In various embodimdents, chemical ligating can be accomplished as disclosed in U.S. Pat. No. 5,476,930 or U.S. Pat. No. 5,681,943

In various configurations, the target probe set can comprise a first target probe comprising a 3′ portion that hybridizes to the small RNA and a 5′ portion having a first PCR primer target sequence, and a second target probe comprising a 5′ portion that hybridizes to the RNA immediately adjacent to the 3′ end of the first target probe and a 3′ portion having a second PCR primer target sequence, wherein the mixture is formed under conditions in which a target probe set hybridizes to the small RNA and ligates to form a probe set ligation sequence. In each target probe, the portion that hybridizes to a small RNA can comprise at least 5 contiguous nucleotides complementary to the small RNA, at least 6 contiguous nucleotides complementary to the small RNA, at least 7 contiguous nucleotides complementary to the small RNA, at least 8 contiguous nucleotides complementary to the small RNA, at least 9 contiguous nucleotides complementary to the small RNA, or at least 10 contiguous nucleotides complementary to the small RNA. In various configurations, at least one target probe, and consequently a probe set, can comprise a detection probe hybridization sequence.

In various embodiments, the 3′ portion of the first target probe that hybridizes to the small RNA and the 5′ portion of the second target probe that hybridizes to the small RNA together can comprise a total of not more than 40 nucleotides, or, in some embodiments, not more than 25 nucleotides. In some configurations, the second target probe can comprise a phosphate moiety at its 5′ end. Thus, a complex comprising a first target probe and a second target probe both hybridized to a small RNA, wherein the 3′ end of the first target probe is immediately adjacent to the 5′ (phosphorylated) end of the second target probe, can serve as a substrate for a ligase, which can ligate the 3′ end of the first target probe to the 5′ end of the second target probe, thereby forming a probe set ligation sequence. Ligation of target probe deoxyribooligonucleotides hybridized to an RNA template can vary in efficiency depending on the sequences selected for the target probes. The sequences of target probes for a small RNA that ligate most efficiently can be determined empirically. For example, a first target probe comprising a 3′-terminal thymidine nucleotide and a second target probe comprising 5′ terminal adenosine nucleotide can ligate more efficiently than other possible target probe pairs when hybridized to a let-7a human microRNA template. Differences in efficiency of ligation can affect amounts of target probe amplification detected. However, in quantitative measurements of a small RNA in a sample, control samples comprising known amounts of a small RNA can be used to produce standard curves to account for efficiencies of ligations.

Probes and primers used in various embodiments of the present invention can be oligodeoxyribonucleotides. These oligodeoxyribonucleotides can be synthesized using routine methods well known to skilled artisans.

The methods of the present invention can further include forming a detection mixture comprising a probe set ligation sequence, a first PCR primer which hybridizes to the complement of the first PCR primer target sequence and a second PCR primer which hybridizes to the second PCR primer target sequence. Any probe set ligation sequence comprised by the detection mixture can be amplified using a polymerase chain reaction, and thereby form an amplification product. A polymerase chain reaction can be conducted using standard conditions well known to skilled artisans. Detection of amplification of any probe ligation sequence can be indicative of a small RNA comprised by the sample.

In various embodiments, detection of amplification can comprise detecting an amplification product of a polymerase chain reaction. Detection of the amplification product can comprise any nucleic acid detection method known to skilled artisans, such as, for example, gel electrophoresis. Gel electrophoresis can use any separation medium, such as an agarose gel or a polyacrylamide gel. Detection can also utilize capillary gel electrophoresis. In certain aspects, one or both PCR primers can comprise a label, such as, for example, a fluorophore or a radioisotope. A label can facilitate detection of an amplification product comprising a labeled PCR primer.

In some embodiments, detection of amplification can comprise detection of a hydrolytic enzyme reaction product. In these configurations, a probe set ligation sequence can comprise a detection probe hybridization sequence, and the enzyme activity in these configurations can be 5′ nuclease activity of a DNA polymerase in a polymerase chain reaction, as described, for example, in U.S. Pat. No. 5,210,015 to Gelfand, U.S. Pat. No. 5,538,848 to and Lie & Petropoulos, Curr. Opin. Biotechnol. 14: 303-308, 1998. In these embodiments, a detection mixture can comprise a detection probe, which can comprise a sequence complementary to a detection probe hybridization sequence.

In various embodiments, a detection method can utilize any probe which can detect a nucleic acid sequence. In some configurations, a detection probe can be, for example, a 5′-exonuclease assay probe such as a TaqMan® probes described herein (see also U.S. Pat. No. 5,538,848), various stem-loop molecular beacons (see e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, e.g., Kubista et al., 2001, SPIE 4264:53-58), non-FRET probes (see, e.g., U.S. Pat. No. 6,150,097), Sunrise®/Amplifluor® probes (U.S. Pat. No. 6,548,250), stem-loop and duplex Scorpion™ probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Labeling probes can also comprise black hole quenchers (Biosearch), Iowa Black (IDT), QSY quencher (Molecular Probes), and Dabsyl and Dabcel sulfonate/carboxylate Quenchers (Epoch). Labeling probes can also comprise two probes, wherein for example a fluorophore is on one probe, and a quencher on the other, wherein hybridization of the two probes together on a target quenches the signal, or wherein hybridization on target alters the signal signature via a change in fluoresence. Labeling probes can also comprise sulfonate derivatives of fluorescein dyes, phosphoramidite forms of fluorescein, phosphoramidite forms of CY5 (available for example from Amersham). In some embodiments, interchelating labels can be used such as ethidium bromide, SYBR® Green I (Molecular Probes), and PicoGreen® (Molecular Probes), thereby allowing visualization in real-time, or end point, of an amplification product in the absence of a labeling probe.

A detection probe in some of these embodiments can further comprise both a fluorophore, and a fluorescence quencher, for example as described in Lee, L. G., et al. Nucl. Acids Res. 21:3761 (1993), and Livak, K. J., et al. PCR Methods and Applications 4: 357 (1995). The fluorescence quencher can be a fluorescent fluorescence quencher, such as the fluorophore TAMRA, or a non-fluorescent fluorescence quencher (NFQ), for example, a combined NFQ-minor groove binder (MGB) such as an MGB Eclipse™ minor groove binder supplied by Epoch Biosciences (Bothell, Wash.) and comprised by TaqMan® probes (Applied Biosystems, Inc.) The fluorophore can be any fluorophore that can be attached to a nucleic acid, such as, for example, FAM, VIC, Sybra Green, TET, HEX, JOE, NED, LIZ, TAMRA, ROX, ALEXA, Texas Red, Cy3, Cy5, Cy7, Cy9, or dR6G. Methods for detecting fluorescence from enzymatic hydrolysis of a fluorogenic probe such as a TaqMan® probe are well known in the art. Upon hybridization of PCR primers and detection probe to a probe set ligation sequence, a DNA polymerase comprising a detection mixture can catalyze hydrolysis of the probe, for example during thermal cycling, and thereby release the fluorophore from inhibition of its fluorescence by the fluorescence quencher. A resulting increase in fluorescence of a fluorophore released from quenching by 5′ nuclease digestion of a fluorogenic probe can be indicative of the presence of a small RNA in a sample.

In various embodiments of the invention, detection of fluorescence of a PCR assay can be by any method known to skilled artisans, and can include, for example, real time detection or end point detection. Detection of fluorescence can be qualitative or quantitative. Quantitative results can be obtained, for example, with the aid of a fluorimeter, for example a fluorimeter comprised by an integerated nucleic acid analysis system, such as, for example, an Applied Biosystems ABI PRISM® 7900HT Sequence Detection System. Furthermore, quantitative results can be obtained in some configurations using a real-time PCR analysis, and determining a threshold cycle for detection of a fluorophore during thermal cycling of a polymerase chain reaction. Some non-limiting examples of protocols for conducting fluorogenic assays such as TaqMan® assays, including analytical methods for performing quantitative assays, can be found in publications such as, for example, “SNPlex™ Genotyping System 48-plex”, Applied Biosystems, 2004; “User Bulletin #2 ABI Prism 7700 Sequence Detection System,” Applied Biosystems 2001; “User Bulletin #5 ABI Prism® 7700 Sequence Detection System,” Applied Biosystems, 2001; and “Essentials of Real Time PCR,” Applied Biosystems (these documents are available on the internet at http://home.appliedbiosystems.com/). Fluorogenic PCR assays used in some configurations of the present invention can be performed using an automated system, such as, for example, an ABI 7700 Sequence Detection System (Applied Biosystems, Foster City, Calif.).

In various embodiments, fluorogenic PCR assays such as TaqMan® assays can be quantified by determining a detection threshold cycle (Ct) value for detection of fluorescence of a fluorogenic polymerase chain reaction, wherein a threshold fluorescence intensity indicative of a signal is selected by an investigator. For a sample of a templated ligation-polymerase chain reaction as described in embodiments of an invention herein, a fluorogenic PCR signal of a sample can be considered to be above background if its Ct value is at least 1 cycle less than that of a no-template control sample.

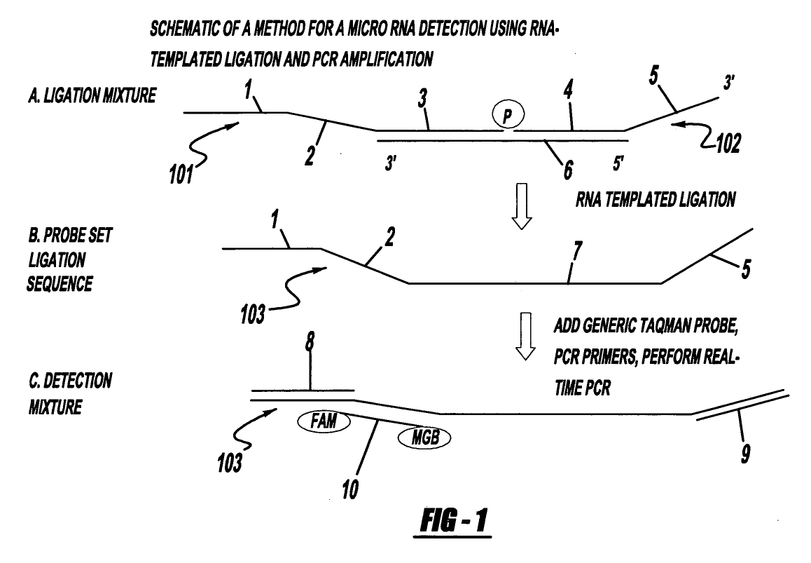

FIG. 1 illustrates one configuration of a small RNA detection assay of the present invention.

In some embodiments, detection of amplification can comprise detection of binding of a detection probe to a detection probe hybridization sequence comprised by a probe set ligation sequence or an amplification product thereof. In some configurations, detecting can comprise contacting a PCR amplification product such as an amplified probe set ligation sequence with a detection probe comprising a label under hybridizing conditions. These detection methods can further comprise separating unhybridized detection probe from a PCR amplification product, and detecting the hybridized detection probe.

In certain configurations, a detection probe hybridization sequence can be a sequence that is comprised by a target probe. In these configurations, a detection mixture can comprise a detection probe in addition to a probe set ligation sequence. A detection probe can comprise a sequence complementary to a sequence comprised by a PCR amplification product, such as a detection probe hybridization sequence. A detection probe hybridization sequence (and its complement) can comprise from about 10 nucleotides up to about 70 nucleotides, from about 12 nucleotides up to about 50 nucleotides, or from about 13 nucleotides up to about 25 nucleotides. In some configurations, a detection probe hybridization sequence can serve as a unique marker for a probe set ligation sequence, and can be, in non-limiting example, complementary to a ZipCode™ sequence (Applied Biosystems), or complementary to a sequence comprised by a TaqMan® probe. In various configurations, a detection probe can further comprise a label, such as a fluorophore or a radioisotope, and can also comprise a mobility modifier. A mobility modifier can be a nucleobase polymer sequence which can increase the size of a detection probe, or in some configurations, a mobility modifier can be a non-nucleobase moiety which increases the frictional coefficient of a probe, such as a mobility modifier described in U.S. Pat. Nos. 5,514,543, 5,580,732, 5,624,800, and 5,470,705 to Grossman. A detection probe comprising a mobility modifier can exhibit a relative mobility in a electrophoretic or chromatographic separation medium that allows a user to identify and distinguish the detection probe from other molecules comprised by a sample. A detection probe comprising a sequence complementary to a detection probe hybridization sequence, such as a ZipCode™ sequence, a fluorphore and a mobility modifier can be, for example, a ZipChute™ probe supplied commercially by Applied Biosystems.

In other embodiments, methods for detecting an RNA can comprise forming a mixture. The mixture can comprise a sample suspected of comprising a small RNA, a target probe set comprising a first target probe and a second target probe as described above, wherein at least one of the first target probe or the second target probe further comprises an affinity tag. The affinity tag can be any moiety which has a binding partner. An affinity tag and its binding partner can have a binding affinity (Ka) of at least about 10⁸ M⁻¹, about 10¹⁰ M⁻¹, about 10¹² M⁻¹, about 10¹⁴ M⁻¹, or about 10¹⁵ M⁻¹. An affinity tag and its binding partner can be, for example, a hapten and an antibody directed against the hapten, or an antigen and an antibody directed against the antigen. For example, the hapten can be biotin or digoxygenin, and the antibody can be a polyclonal antibody or a monoclonal antibody directed against biotin or digoxygenin. In some configurations, a binding partner can be a non-antibody binding partner, such as streptavidin or avidin polypeptides as a binding partner for biotin. In addition, a mixture can comprise an affinity tag binding partner which can be covalently attached to a solid phase support. Upon formation of the mixture, a complex can form comprising the solid phase support, the affinity tag binding partner, a first and second target probe, at least one of which comprises the affinity tag, and the small RNA hybridized to the target probes. A solid phase support can comprise, for example, beads covalently attached to a binding partner, for example beads covalently attached to streptavidin. Such beads can be, in some configurations, paramagnetic beads. In some configurations, a mixture comprising a complex bound to a solid phase support can be washed, thereby removing first target probe and second target probe that is not hybridized to the small RNA. Washing the mixture can comprise subjecting the complex to one or more cycles of suspension in a buffer. Without being limited by theory, it is believed that a ligase such as T4 ligase can artificially ligate oligonucleotides in vitro in the absence of a hybridization template. Such artificial ligation products can cause background signals to develop in subsequent PCR amplifications. Washing the mixture can remove from a mixture target probe sequences that are not bound to a small RNA. A ligase such as a T4 ligase can be added to a washed mixture comprising a complex bound to the solid support to form a probe set ligation sequence, which can be bound to the solid support. Amplification by PCR of a probe set ligation sequence can therefore occur with little or no artificially ligated target probes to cause formation of background amplification products. Hence, the disclosed methods can be sufficiently sensitive to detect less than 1 attomole of a small RNA in a sample, and as few as 60,000 copies of a small RNA or as few as 120,000 copies of a small RNA in these embodiments of the invention.

In various embodiments, the present invention can include methods for detecting a plurality of small RNAs in a sample. The method can comprise forming a ligation mixture that comprises (a) a sample suspected of comprising the plurality of small RNAs; (b) a ligase; and (c) a plurality of target probe sets, each target probe set comprising a first target probe and a second target probe as described above. In these embodiments, each target probe set ligation sequence can comprise a unique detection probe hybridization sequence, such as, for example, a ZipCode™ sequence described above. The detection mixture can be formed comprising a plurality of probe set ligation sequences, a first PCR primer which hybridizes to the complement of the first PCR primer targets, a second PCR primer which hybridizes to the second PCR primer targets, and a DNA polymerase. In some configurations, the first PCR primer target sequence can be the same sequence for all first target probes; the second PCR primer target sequence can be the same sequence for all second target probes; and the first and second PCR primers can be the same first and second primers for amplification of all the probe set ligation sequences comprised by a detection mixture. Each probe set ligation sequence can be amplified using a polymerase chain reaction using the first and second PCR primers. Detection of amplification of any probe ligation sequence can be indicative of a small RNA comprised by the sample. In some configurations of these embodiments, a plurality of detection probes can detect a plurality of detection probe hybridization sequences such as ZipCode™ sequences described above.

The invention also provides, in various embodiments, additional methods of detecting a plurality of small RNAs. Methods of these embodiments can comprise forming a hybridization mixture comprising (a) a sample suspected of comprising a plurality of small RNAs, (b) a plurality of target probe sets for detecting the small RNA, each target probe set comprising (i) a first target probe and a second target as described above in which at least one of the first target probe and the second target probe further comprises an affinity tag, and wherein at least one of the first target probe and the second target probe further comprises a detection probe hybridization sequence, and (c) a binding partner for the affinity tag bound to a solid support, wherein the mixture is formed under conditions in which a target probe set hybridizes to a small RNA. The affinity tag and its binding partner can be as described above. In these embodiments, a plurality of complexes can form, each complex comprising a first and second target probe, a small RNA, and an affinity tag binding partner bound to a solid support. This mixture can be washed as described above to remove first and second target probes which are not hybridized to a small RNA. The washed mixture can then be contacted a ligase such as a T4 ligase to form a plurality of probe set ligation sequences. In various configurations, a detection mixture can be formed, comprising the plurality of probe set ligation sequences, a first PCR primer which hybridizes to the complement of each first PCR primer targets, a second PCR primer which hybridizes to each second PCR primer targets, and a DNA polymerase. As above, the first primer targets can all comprise the same sequence and the second primer targets can all comprise the same sequence. The first and second PCR primers can therefore be “universal” primers that can amplify any probe set ligation sequence comprised by the detection mixture in a polymerase chain reaction. Small RNAs comprised by a sample can be detected by amplification of probe set ligation sequences using standard PCR conditions well known to skilled artisans.

Amplification of any probe set ligation sequence comprised by the detection mixture can be detected by any detection method known to a skilled artisan. In some embodiments, a PCR probe can comprise a label, such as a fluorophore or a radioisotope. In these embodiments, determining the identity of a small RNA comprised by a detection mixture can comprise contacting an array comprising a plurality of loci with the detection mixture under hybridization conditions, wherein each locus comprises a detection probe which comprises a sequence complementary to a detection probe hybridization sequence comprised by an amplification. The detection probe sequences can be, for example, ZipCode™ sequences or their complements, as described above. The presence or quantity of a small RNA in a sample can be determined by detecting the hybridization of a label comprised by a PCR probe at an identified locus comprised by the array.

In various embodiments, detection of a plurality of small RNAs can comprise contacting a detection mixture with a plurality of detection probes under hybridization conditions, wherein each detection probe comprises a label, such as a fluorophore, a radioisotope or a mobility modifier. In some configurations, amplified sequences of a detection mixture can be bound to a solid support, for example through the use of an affinity tag. Determining the identity of a small RNA comprised by the detection mixture can comprise washing the amplified sequences to remove unhybridized detection probe, and determining the identity of the remaining detection probes by detecting the label(s) comprised by probes. For example, each detection probe can comprise a unique fluorophore or a unique mobility modifier.

In certain configurations, the second target probe can be phosphorylated at its 5′ end with a kinase, such as, for example, SNPlex kinase, supplied commercially by Applied Biosystems.

In related embodiments, amplification of a plurality of ligated primer sets can also be detected using a plurality of 5′ nuclease assays, as described above. Each of the probes in these assays can comprise a different fluorophore.

In various other embodiments, the present invention can include a kit for the detection of a small RNA. The kit can comprise a first target probe comprising a 3′ portion that hybridizes to a small RNA and a 5′ portion having a first PCR primer target sequence and a second target probe comprising a 5′ portion that hybridizes to the small RNA immediately adjacent to the 3′ portion of the first target probe and a 3′ portion having a second PCR primer target sequence, wherein upon hybridization of the first and second target probes to the small RNA in the presence of a ligase, a ligation sequence can be formed. In some configurations, components of the kit can be packaged in a container.

In various configurations, a small RNA detected with the aid of a kit of the present invention can comprise from about 10 to about 40 contiguous nucleotides, or from about 20 to about 30 contiguous nucleotides.

In various configurations, at least one target probe can further comprise a detection probe hybridization sequence. The sequence can be, for example, a ZipCode™ sequence or its complement, as described above. In certain configurations, a detection probe hybridization sequence can by a sequence complementary to a 5′ nuclease detection assay probe, such as a TaqMan® probe described above. The kit can further comprise a detection probe comprising a sequence complementary to a detection probe hybridization sequence. The detection probe can be, for example, a ZipChute™ probe described above, or a TaqMan® probe described above. The kit component can comprise a fluorophore such as, in non-limiting examples, FAM, VIC, Sybra Green, TET, HEX, JOE, NED, LIZ, TAMRA, ROX, ALEXA, Texas Red, Cy3, Cy5, Cy7, Cy9, and/or dR6G. A TaqMan® probe can further comprise a fluorescence quencher, such as TAMRA or a non-fluorescent fluoescence quencher, such as a minor groove binder (NFQ-MGB) supplied by Epoch Biosciences.

A kit of these embodiments can comprise target probes wherein the 3′ portion of the first target probe that hybridizes to the small RNA and the 5′ portion of the second target probe that hybridizes to the small RNA together have a total of not more than about 40 nucleotides, or not more than about 25 nucleotides.

The small RNA can be, in non-limiting example, an mRNA, an siRNA, an stRNA or an snRNA. Some non-limiting examples of a small RNA in these configurations can be let-7a, miR-16, miR-20, and miR-30.

In some configurations, at least one of the first target probe or the second target probe can further comprise an affinity tag, such as, for example, biotin or digoxygenin. In some configurations, a kit can comprise a binding partner for an affinity tag, such as, for example, an antibody directed against the affinity tag, or an avidin or a streptavidin. An affinity tag binding partner can be covalently bound to a solid phase support. A solid phase support can comprise a plurality of paramagnetic beads.

A kit of these embodiments can also comprise a ligase that can ligate oligonucleotides hybridized to an RNA template. For example, a kit can comprise a ligase of a T4 bacteriophage (“T4 ligase”).

In certain embodiments, the detection probe comprised by a kit can comprise an electrophoretic mobility modifier, as described above.

In certain embodiments, the kit can comprise a plurality of probe sets wherein at least one target probe of each of the plurality of probe sets comprises a unique identifier sequence, such as a ZipCode™ sequence or its complement.

In various embodiments, the kit can further comprise an array comprising a plurality of detection loci, wherein each detection locus comprises a hybridization probe that uniquely hybridizes to each unique identifier sequence.

The invention can be further understood by reference to the examples which follow.

### EXAMPLE 1

This example illustrates a schematic for a method for detection of a microRNA in a sample, as diagrammed in FIG. 1, as follows.

A. A ligation mixture is formed comprising a first target probe, a second target probe, and a target microRNA comprised by a sample. The first target probe (101) is a deoxyribooligonucleotide comprising, in 5′ to 3′ direction, a first PCR primer sequence (1), a detection probe hybridization sequence, in this case a TaqMan® probe hybridization sequence (2), and a sequence complementary to a 3′ portion of a target microRNA (3). The second target probe (102) is a deoxyribooligonucleotide comprising, in the 5′ to 3′ direction, a 5′-terminal phosphate, a sequence complementary to a 5′ portion of the target microRNA and which hybridizes to the microRNA immediately adjacent to the 3′ terminal of the first target probe (4), and a sequence complementary to a second PCR primer (5). The target microRNA (6) is hybridized throughout its length with a combination of sequences comprised by the first target probe and the second target probe.

B. Upon treatment of the ligation mixture with T4 ligase, a probe set ligation sequence is formed, comprising, in the 5′ to 3′ direction, a first PCR primer sequence (1), a detection probe hybridization sequence (2), a sequence complementary to the target microRNA (7), a sequence complementary to a 5′ portion of the target microRNA, and a sequence complementary to a second PCR primer (5).

C. A detection mixture comprises the probe set ligation sequence (103), a first PCR primer (8), a second PCR primer (9) and a fluorogenic TaqMan® probe (10) comprising the fluorophore FAM and an NFQ-MGB fluorescence quencher. The mixture can be subjected to thermal cycling. Fluorescence of the fluorophore comprised by the TaqMan® probe increases during thermal cycling as the taq polymerase digests the TaqMan® probe, releasing the fluorophore from quenching by the non-fluorescent quencher-minor groove binder. Intensity of the fluorescence is proportional to the amount of the microRNA in the sample analyzed, and the number of PCR cycles. The amount of microRNA comprised by the sample is calculated by determining a threshold cycle (Ct) value for detection, compared to threshold cycle (Ct) values of control samples of known microRNA content.

### EXAMPLE 2

This example illustrates a time course of RNA-templated oligonucleotide ligation using T4 DNA ligase and four different synthetic mRNAs.

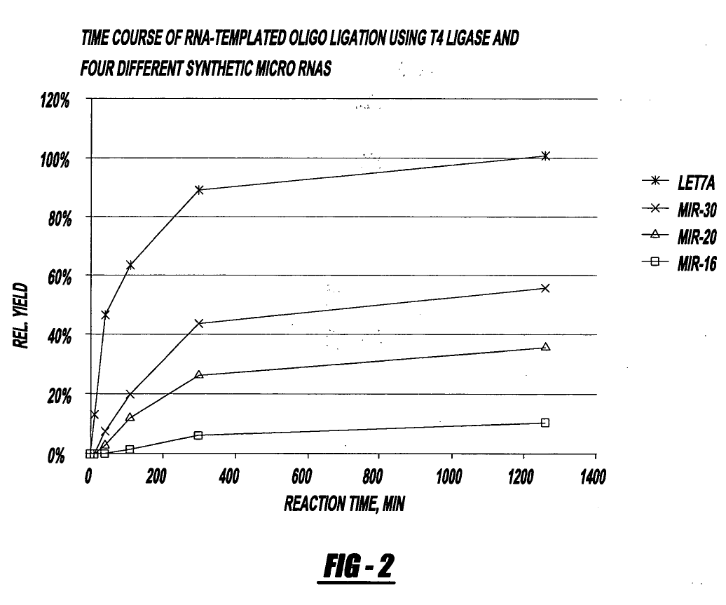

In this example, as shown in FIG. 2, four different mRNAs were utilized as templates for ligation of deoxyribonucleotides. The mRNAs were those listed in Table 2.

TABLE 2

| | | | |
|---|---|---|---|
| | | Identi- | Reaction |
| miRNA | Sequence | fication | velocity |

| | | | |
|---|---|---|---|
| let-7a | UGAGGUAGUAGGUUGUAUAUU | SEQ ID NO: 1 | 26 |
| | | | fmol/min |
| miR-30 | CUUUCAGUCGGAUGUUUGCAGC | SEQ ID NO: 24 | 3.7 |
| | | | fmol/min |
| miR-20 | UAAAGUGCUUAUAGUGCAGGUA | SEQ ID NO: 13 | 2.6 |
| | | | fmol/min |
| miR-16 | UAGCAGCACGUAAAUAUUGGCG | SEQ ID NO: 8 | 0.5 |
| | | | fmol/min |

The first and second deoxyribooligonucleotide probes comprised the sequences listed in tables 3 and 4, respectively:

TABLE 3

| | | |
|---|---|---|
| miRNA | First probe | Identification |

| | | |
|---|---|---|
| let-7a | AACTATACAACCT | SEQ ID NO: 32 |
| miR-30 | GCTGCAAACAT | SEQ ID NO: 33 |
| miR-20 | TACCTGCACTAT | SEQ ID NO: 34 |
| miR-16 | CGCCAATATTT | SEQ ID NO: 35 |

TABLE 4

| | | |
|---|---|---|
| miRNA | Second probe | Identification |

| | | |
|---|---|---|
| let-7a | ACTACCTCA | SEQ ID NO: 36 |
| miR-30 | CCGACTGAAAG | SEQ ID NO: 37 |
| miR-20 | AAGCACTTTA | SEQ ID NO: 38 |
| miR-16 | ACGTGCTGCTA | SEQ ID NO: 39 |

In these experiments, the following reaction conditions were used: 50 microliter reactions each containing 40 nM synthetic mRNA, first and second target probes, and 15 Weiss units T4 ligase (1800 nM) were incubated at 30° C. Aliquots were withdrawn at indicated timepoints, and reactions stopped by the addition of 5 microliters of 100 mM EDTA and chilling on ice. Aliquots were diluted 1:100 prior to analysis on an ABI 3100 system. The results indicate that all of the mRNAs tested could serve as template for RNA ligation, albeit reaction rates differed for different mRNAs.

### EXAMPLE 3

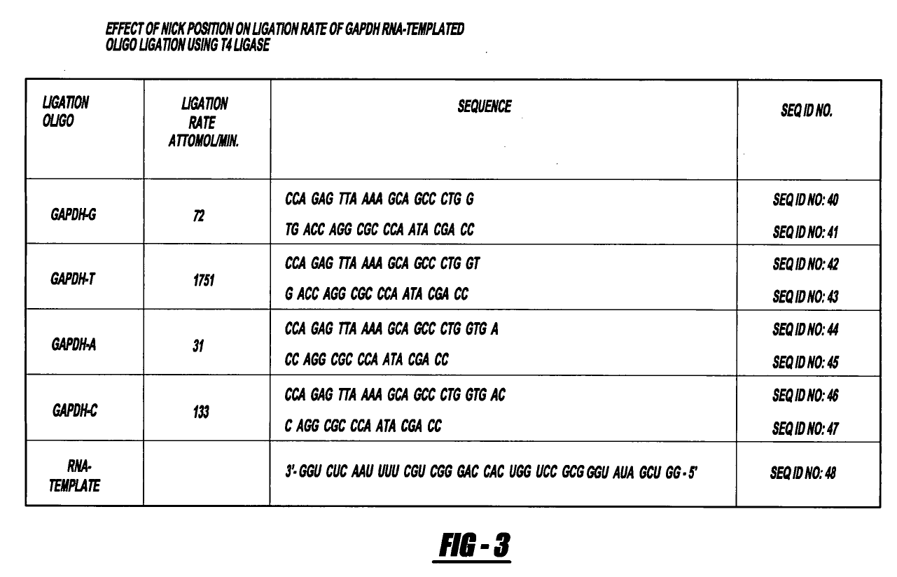

This example illustrates ligation rates for a variety of deoxyribooligonucleotide pairs hybridized to a 44 nucleotide RNA template. The probe pairs all hybridized to the same sequence, but the nucleotides at the 3′ ends of the first target probes and the 5′ ends of the second target probes differed for the different probe pairs. The samples were treated as in Example 2 and the results are reported in FIG. 3. The efficiency of ligation varied over an approx. 56-fold range for the target pairs ligating using the GAPDH RNA template.

### EXAMPLE 4

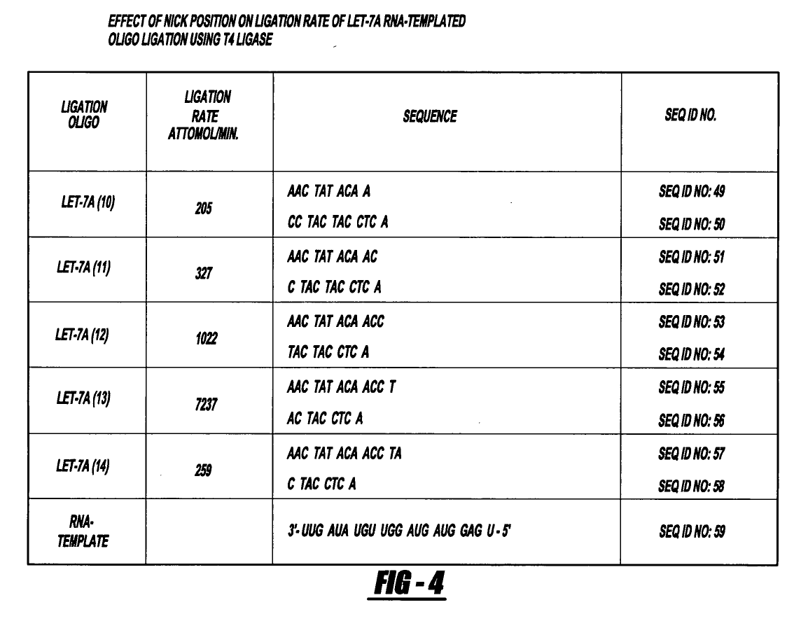

This example illustrates ligation rates of let-7a micro RNA-templated ligation. The probe pairs all hybridized to the same sequence, but the nucleotides at the 3′ ends of the first target probes and the 5′ ends of the second target probes differed for the different probe pairs. The samples were treated as in Example 2 and the results are reported in FIG. 4. The efficiency of ligation varied over an approx. 35-fold range for the target pairs ligating using the lit-7a RNA template.

### EXAMPLE 5

This example illustrates detection of micro RNA miR-20 using mRNA-templated oligonucleotide ligation and PCR amplification. In these experiments, 20 ul ligation reactions contained 5 Weiss U of T4 DNA ligase (Takara) (750 nM) in a ligation buffer, no ATP, 10 nM deoxyribooligonucleotide target probe pairs, and mRNA synthetic RNA template target ranging from 0 (no template control, NTC) to 30 million copies of miR-20 RNA artificial template (22 nucleotides). The template RNA and deoxyribooligonucleotide sequences are shown in table 5:

TABLE 5

| Nucleic | | |
|---|---|---|
| acid | Sequence | Indentification |
| miR-20 | UAAAGUGCUUAUAGUGCAGGUA | SEQ ID NO: 13 |
| (RNA) | | |
| MiR-20 | TTGCCTGCTCGACTTAGATTTTTTGC | SEQ ID NO: 59 |
| first | ACTGCCAAGACTTACCTGCACTAT | |
| target | | |
| probe | | |
| MiR-20 | 5′phosphate- | SEQ ID NO: 60 |
| second | AAGGACTTTAATCACTGGATAGCGA | |
| target | CGTGG | |
| probe | | |
| First | TTGCCTGCTCGACTTAGA | SEQ ID NO: 61 |
| PCR | | |
| primer | | |
| Second | CCACGTCGCTATCCAGTGAT | SEQ ID NO: 62 |
| PCR | | |
| primer | | |
| TaqMan ® | 6Fam-TGCACTGCCAAGACT-NFQ- | SEQ ID NO: 63 |
| probe | MGB | |

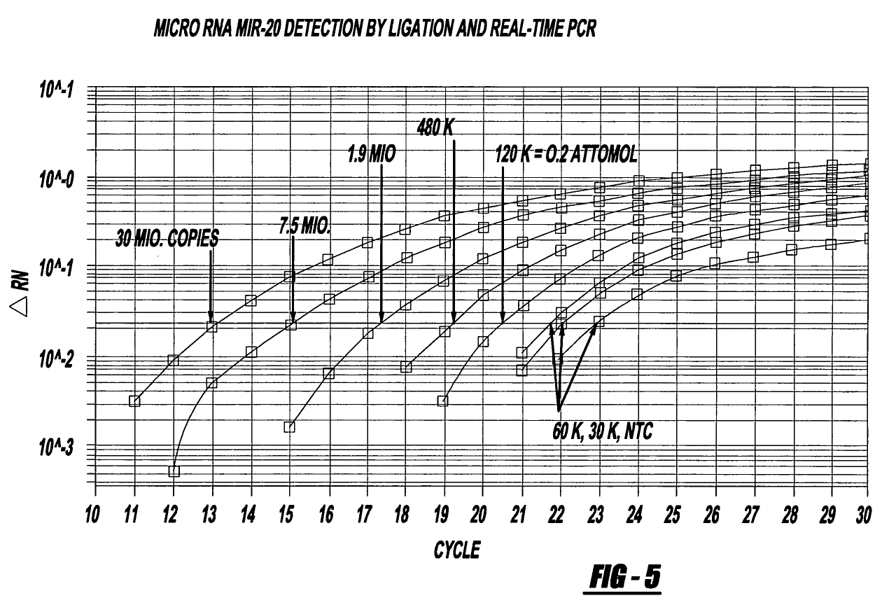

Reactions were incubated overnight at 30° C. 1/10th vol. (2 ul) of the ligation reaction was used per 20 microliter TaqMan® real-time PCR amplification reaction using the 5′-tails of the ligation oligos. Fluorescence intensities were recorded for each cycle of the PCR. As shown in FIG. 5, samples comprising 30,000 copies, 60,000 copies, 120,000 copies (or more) of the miR-20 micro RNA could be detected using real time PCR, in that the detection threshold cycles (Ct) are less than the detection threshold cycle for a no template control.

### EXAMPLE 6

This example illustrates detection of micro RNA miR-20 using mRNA-templated oligonucleotide ligation and PCR amplification. In these experiments, 20 ul ligation reactions contained 5 Weiss U of T4 DNA ligase (Takara) (1500 nM) in a ligation buffer, no ATP, 10 nM deoxyribooligonucleotide target probe pairs, 0 to 50 ng of HeLa total RNA (Ambion), 0 to 30 million copies of miR-20 RNA artificial template (22 nucleotides). The template RNA and deoxyribooligonucleotide sequences are shown in table 5, supra.

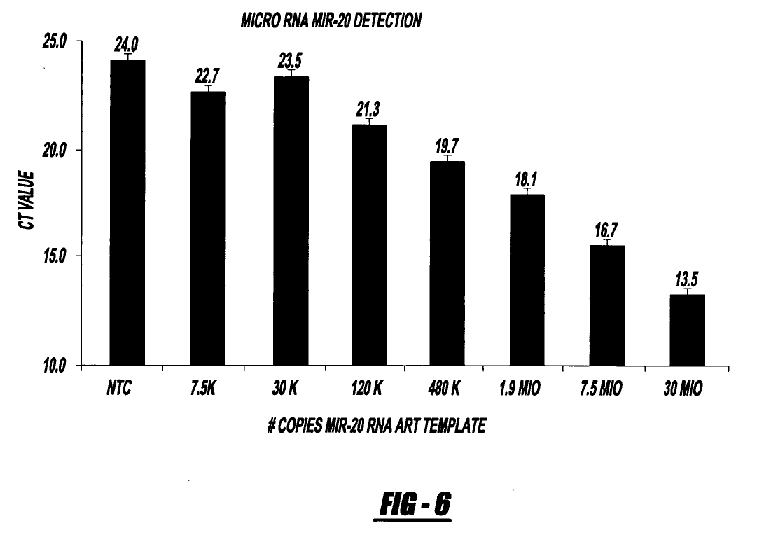

Reactions were incubated overnight at 30° C. 1/10th vol. (2 ul) of the ligation reaction was used per 20 microliter TaqMan® real-time PCR amplification reaction using the PCR primer hybridization sequences of the target detection deoxyribooligonucleotides for amplification. Fluorescence intensities were recorded for each cycle of the PCR. As shown in FIG. 6, samples comprising 120,000 copies (or more) of the miR-20 micro RNA could be detected using real time PCR, in that the detection threshold cycle (Ct) is less than the detection threshold cycle for a no template control.

### EXAMPLE 7

This example illustrates detection of microRNAs let-7a and miR-16 in HeLa cells using RNA-templated ligation and PCR.

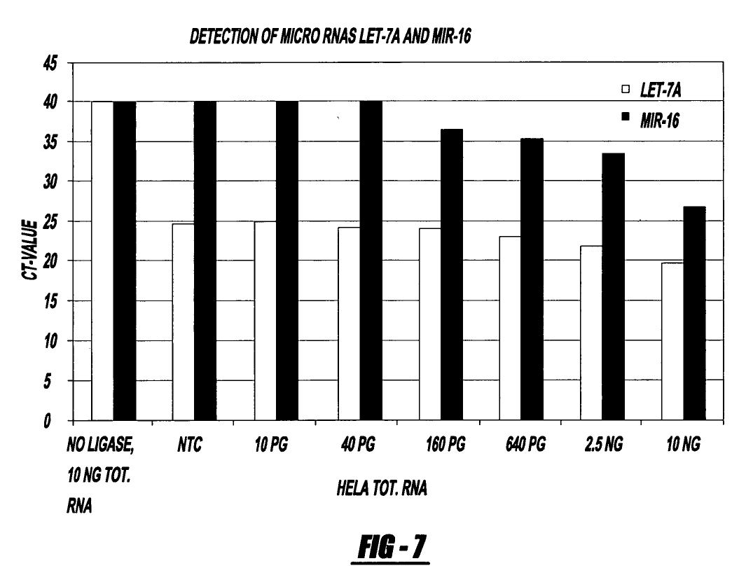

In these experiments, RNA from HeLa cells in indicated amounts was used as template for ligations using deoxyribonucleotide probes for mRNAs let-7a and miR-16. The following conditions were used: 20 μl ligation reaction mixtures comprised the indicated amount of HeLa cell total RNA; 2.5 Weiss U (Roche) T4 ligase in a ligation buffer (no ATP); 2.5 nM each deoxyribonucleotide probe. Reaction mixtures were incubated 4 hours at 20° C. 2 μl of each ligation reaction mixture was used as target in real-time TaqMan® assays using 20 μl reactions comprising PCR primers directed to common first and common second PCR primer targets in the deoxyribonucleotide probes. The results, shown in FIG. 7, indicate that detection threshold cycle (Ct) values decreased with increasing amounts of HeLa RNA. Hence, both mRNAs could be detected in the samples comprising at least 160 pg of HeLa RNA.

### EXAMPLE 8

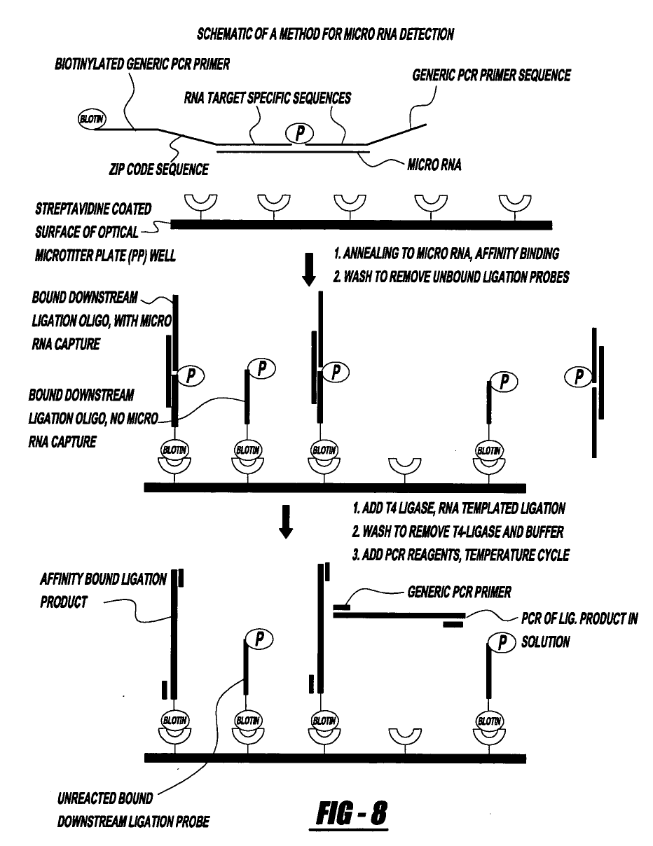

This example illustrates a schematic for a method for detection of a microRNA in a sample, as diagrammed in FIG. 8.

In this example, a first target probe comprises an affinity tag, in this case biotin. The first probe further comprises a first PCR primer sequence, a detection probe hybridization sequence (in this case a ZipCode™ sequence), and a first sequence that hybridizes to a microRNA. A second target probe comprises a 5′ phosphate, a sequence that hybridizes to the microRNA immediately adjacent to the first target probe, and a second PCR primer sequence. The probes form a complex with the mRNA and an affinity tag binding partner, in this case a streptavidin bound to a solid support in this case an optical microtiter plate.

### EXAMPLE 9

This example illustrates screening for ligation reaction additives.

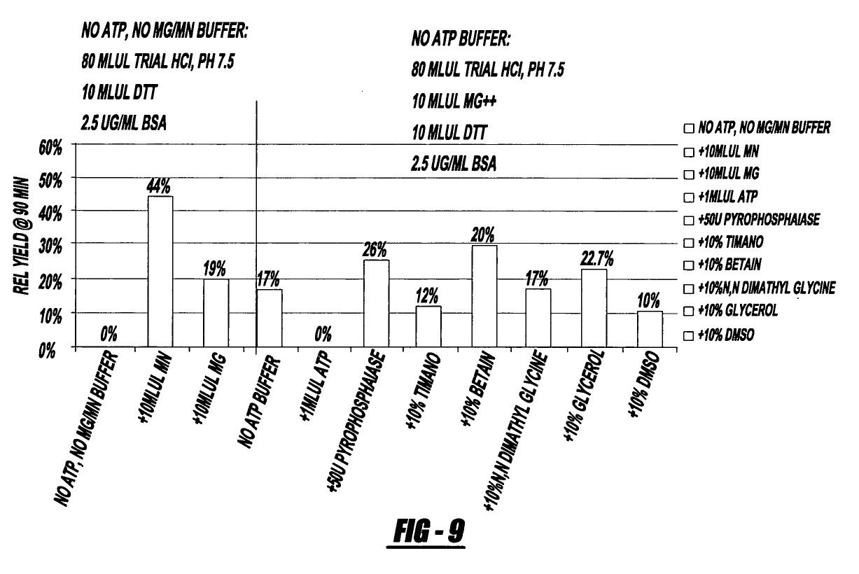

In this example, several additives that had been shown to alter enzyme kinetics of DNA polymerase reactions such as PCR or DNA sequencing reactions, were screened for their effects upon RNA-templated deoxyribooligonucleotide ligation. In these experiments, the following reaction conditions were used: 40 μl reactions containing 40 nM synthetic micro RNA miR-20, first and second target probes, the second target probe comprising a 3′FAM fluorophore, and 6 Weiss U of T4 ligase (900 nM) were incubated at 30° C. for 90 min. Reactions were stopped by combining 5 μl aliquots with 5 μl 100 mM EDTA and chilling on ice. Samples were diluted 100-fold then analyzed on an ABI 3100 sequence detection system. As shown in FIG. 9, it was found that (a) the addition of betaine 10% (0.85 M) and (b) the substitution of Mg⁺² with Mn⁺² both enhanced ligation reaction rates. Other reaction condition variations which altered ligation reaction rates are also shown in FIG. 9.

All references cited in this specification are hereby incorporated by reference. Any discussion of references cited herein is intended merely to summarize the assertions made by their authors and no admission is made that any reference or portion thereof constitutes relevant prior art. Applicants reserve the right to challenge the accuracy and pertinency of the cited references.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A method for detecting a small RNA, the method comprising:
(1) forming a mixture comprising (a) a sample suspected of comprising the small RNA; (b) a ligating agent; and (c) a target probe set for detecting the small RNA, the target probe set comprising (i) a first target probe comprising a 3′ portion that hybridizes to the small RNA and a 5′ portion having a first PCR primer target sequence, and (ii) a second target probe comprising a 5′ portion that hybridizes to the RNA immediately adjacent to the 3′ end of the first target probe and a 3′ portion having a second PCR primer target sequence, wherein the mixture is formed under conditions in which a target probe set hybridizes to the RNA and ligates to form a probe set ligation sequence;
(2) forming a detection mixture comprising a probe set ligation sequence, a first PCR primer which hybridizes to the complement of the first PCR primer target, and a second PCR primer which hybridizes to the second PCR primer target;
(3) amplifying any probe set ligation sequence comprised by the detection mixture using a polymerase chain reaction; and
(4) detecting amplification of any probe set ligation sequence comprised by the detection mixture,
wherein the small RNA comprises a sequence consisting of from about 20 to about 40 contiguous nucleotides.

2. A method according to claim 1, wherein the small RNA comprises from about 20 to about 30 contiguous nucleotides.

3. A method according to claim 1, wherein the small RNA is an mRNA, an siRNA, or an snRNA.

4. A method according to claim 3, wherein the small RNA is selected from the group consisting of let-7a, miR-16, miR-20, and miR-30.

5. A method according to claim 1, wherein the 3′ portion of the first target probe that hybridizes to the small RNA and the 5′ portion of the second target probe that hybridizes to the small RNA together have a total of not more than about 40 nucleotides.

6. A method according to claim 5, wherein the 3′ portion of the first target probe that hybridizes to the small RNA and the 5′ portion of the second target probe that hybridizes to the small RNA together have a total of not more than about 25 nucleotides.

7. A method according to claim 1, wherein at least one target probe further comprises a detection probe hybridization sequence, and wherein the detection mixture further comprises a detection probe comprising a sequence that hybridizes to the detection probe hybridization sequence and a fluorophore, and wherein the detecting is by a fluorescence assay.

8. A method according to claim 7, wherein the 5′ nuclease detection assay is a real-time PCR assay.

9. A method according to claim 7, wherein the 5′ nuclease detection assay is an end-point PCR assay.

10. A method according to claim 7, wherein the fluorophore is selected from the group consisting of FAM, VIC, Sybra Green, TET, HEX, JOE, NED, LIZ, TAMRA, ROX, ALEXA, Texas Red, Cy3, Cy5, Cy7, Cy9, and dR6G.

11. A method according to claim 1, wherein at least one target probe further comprises a detection probe hybridization sequence, and at least one PCR primer comprises a label, and wherein the detecting comprises (i) contacting the detection mixture with an array comprising a plurality of detection loci in which each detection locus comprises a detection probe that hybridizes to the detection probe hybridization sequence, and (ii) detecting the label at a locus.

12. A method according to claim 11, wherein the label is a fluorophore selected from the group consisting of FAM, VIC, Sybra Green, TET, HEX, JOE, NED, LIZ, TAMRA, ROX, ALEXA, Texas Red, Cy3, Cy5, Cy7, Cy9, and dR6G.

13. A method according to claim 1, wherein at least one target probe further comprises a detection probe hybridization sequence, and wherein the detection mixture further comprises a detection probe comprising a sequence that hybridizes to the detection probe hybridization sequence, a label and an electrophoretic mobility modifier, and the detecting comprises detecting hybridization of the detection probe to the detection probe hybridization sequence by a gel electrophoresis assay.

14. A method according to claim 11, wherein the label is a fluorophore selected from the group consisting of FAM, VIC, Sybra Green, TET, HEX, JOE, NED, LIZ, TAMRA, ROX, ALEXA, Texas Red, Cy3, Cy5, Cy7, Cy9, and dR6G.

15. A method according to claim 1, wherein the ligating agent is a ligase.

16. A method according to claim 15, wherein the ligase is a T4 ligase.

17. A method according to claim 1, wherein the ligating agent is at least one of a chemical ligating agent comprising a phosphorothioate, a tosylate or iodide group.

18. A method according to claim 1, wherein the ligating agent is selected from the group consisting of carbodiimide, cyanogen bromide (BrCN), imidazole, 1-methylimidazole/carbodiimide/cystamine, N-cyanoimidazole, dithiothreitol (DTT) and ultraviolet light.

19. A method for detecting a small RNA in a sample, the method comprising:
(1) forming a mixture comprising (a) a sample suspected of comprising the small RNA; (b) a target probe set for detecting the small RNA, the target probe set comprising (i) a first target probe comprising a 3′ portion that hybridizes to the small RNA and a 5′ portion having a first PCR primer target sequence, and (ii) a second target probe comprising a 5′ portion that hybridizes to the RNA immediately adjacent to the 3′ end of the first target probe and a 3′ portion having a second PCR primer target sequence, wherein one of the first or second target probes further comprises an affinity tag; and (c) an affinity tag binding partner which is covalently attached to a solid phase support, wherein upon forming the mixture, a complex forms comprising the small RNA hybridized to the first target probe and the second target probe, and the affinity tag binding partner bound to the affinity tag;
(2) removing unhybridized first target probe and unhybridized second target probe from the mixture;
(3) forming a probe set ligation sequence by contacting the complex with a ligating agent;
(4) forming a detection mixture comprising the probe set ligation sequence, a first PCR primer which hybridizes to the complement of the first PCR primer target, and a second PCR primer which hybridizes to the second PCR primer target;
(5) amplifying any probe set ligation sequence; and
(6) detecting amplification of any probe set ligation sequence comprised by the mixture.

20. A method according to claim 19, wherein the method detects less than 1 attomole of the small RNA in the sample.

21. A method according to claim 19, wherein the method detects as few as 60,000 copies of the small RNA in the sample.

22. A method according to claim 19, wherein the method detects as few as 120,000 copies of the small RNA in the sample.

23. A method according to claim 19, wherein the affinity tag is selected from the group consisting of biotin and digoxygenin.

24. A method according to claim 19, wherein the affinity tag binding partner is selected from the group consisting of streptavidin, avidin, an antibody directed against biotin and an antibody directed against digoxygenin.

25. A method according to claim 19, wherein the solid phase support comprises a plurality of paramagnetic beads.

26. A method according to claim 19, wherein the small RNA comprises from about 20 to about 30 contiguous nucleotides.

27. A method according to claim 19, wherein the small RNA is a mRNA, a siRNA, or a snRNA.

28. A method according to claim 24, wherein the small RNA is selected from the group consisting of let-7a, miR-16, miR-20, and miR-30.

29. A method according to claim 19, wherein the 3′ portion of the first target probe that hybridizes to the small RNA and the 5′ portion of the second target probe that hybridizes to the small RNA together have a total of not more than about 40 nucleotides.

30. A method according to claim 19, wherein the 3′ portion of the first target probe that hybridizes to the small RNA and the 5′ portion of the second target probe that hybridizes to the small RNA together have a total of not more than about 25 nucleotides.

31. A method according to claim 19, wherein at least one target probe further comprises a detection probe hybridization sequence, and wherein the detection mixture further comprises a detection probe.

32. A method according to claim 28, wherein the detection probe is a 5′-exonuclease assay probe

33. A method according to claim 31, wherein the detecting amplification comprises a real-time PCR assay.

34. A method according to claim 31, wherein the detecting amplification comprises an end-point PCR assay.

35. A method according to claim 19, wherein at least one target probe further comprises a detection probe hybridization sequence, and at least one PCR primer comprises a label, and wherein the detecting comprises (i) contacting the detection mixture with an array comprising a plurality of detection loci in which each detection locus comprises a detection probe that hybridizes to the detection probe hybridization sequence, and (ii) detecting the fluorophore at a locus.

36. A method for detecting small RNA according to claim 35, wherein the label is a fluorophore selected from the group consisting FAM, VIC, Sybra Green, TET, HEX, JOE, NED, LIZ, TAMRA, ROX, ALEXA, Texas Red, Cy3, Cy5, Cy7, Cy9, and dR6G.

37. A method according to claim 19, wherein at least one target probe further comprises a detection probe hybridization sequence, and wherein the detection mixture further comprises a detection probe comprising a sequence that hybridizes to the detection probe hybridization sequence, a label and an electrophoretic mobility modifier, and the detecting comprises detecting hybridization of the detection probe to the detection probe hybridization sequence by a gel electrophoresis assay.

38. A method according to claim 37, wherein the label is a fluorophore selected from the group consisting FAM, VIC, Sybra Green, TET, HEX, JOE, NED, LIZ, TAMRA, ROX, ALEXA, Texas Red, Cy3, Cy5, Cy7, Cy9, and dR6G.

39. A method according to claim 19, wherein the ligase is T4 ligase.

40. A small RNA detection kit comprising:
a first target probe comprising a 3′ portion that hybridizes to a small RNA and a 5′ portion having a first PCR primer target sequence;
a second target probe comprising a 5′ portion that hybridizes to the small RNA immediately adjacent to the 3′ portion of the first target probe and a 3′ portion having a second PCR primer target sequence, wherein upon hybridization of the first and second target probes to the small RNA in the presence of a ligase, a ligation sequence is formed, packaged in a container.

41. A kit according to claim 40, wherein one of the first or second target probes further comprises an affinity tag.

42. A kit according to claim 40, wherein the affinity tag is selected from the group consisting of biotin and digoxygenin.

43. A kit according to claim 40, further comprising an affinity tag binding partner attached to a solid phase support.

44. A kit according to claim 43, wherein the affinity tag binding partner is selected from the group consisting of streptavidin, avidin, an antibody directed against biotin and an antibody directed against digoxygenin.

45. A kit according to claim 43, wherein the solid phase support comprises a plurality of paramagnetic beads.

46. A kit according to claim 43, wherein the small RNA comprises from about 20 to about 30 contiguous nucleotides.

47. A kit according to claim 40, wherein the small RNA is a mRNA, a siRNA, or a snRNA.

48. A kit according to claim 47, wherein the small RNA is selected from the group consisting of let-7a, miR-16, miR-20, and miR-30.

49. A kit according to claim 40, wherein the 3′ portion of the first target probe that hybridizes to the small RNA and the 5′ portion of the second target probe that hybridizes to the small RNA together have a total of not more than about 40 nucleotides.

50. A kit according to claim 49, wherein the 3′ portion of the first target probe that hybridizes to the small RNA and the 5′ portion of the second target probe that hybridizes to the small RNA together have a total of not more than about 25 nucleotides.

51. A kit according to claim 40, wherein at least one target probe further comprises a detection probe hybridization sequence.

52. A kit according to claim 51, further comprising a detection probe which hybridizes to the detection probe hybridization sequence.

53. A kit according to claim 52, wherein the detection probe comprises a label.

54. A kit according to claim 53, wherein the label is a fluorophore selected from the group consisting of FAM, VIC, Sybra Green, TET, HEX, JOE, NED, LIZ, TAMRA, ROX, ALEXA, Texas Red, Cy3, Cy5, Cy7, Cy9, and dR6G.

55. A kit according to claim 54, wherein the detection probe further comprises a fluorescence quencher.

56. A kit according to claim 40, further comprising a ligase.

57. A kit according to claim 56, wherein the ligase is T4 ligase.

58. A kit according to claim 52, wherein the detection probe comprises an electrophoretic mobility modifier.

59. A kit according to claim 40, wherein the first and second target probes comprise a target probe set and the kit comprises a plurality of probe sets, wherein at least one target probe of each of the plurality of probe sets comprises a unique identifier sequence.

60. A kit according to claim 59, further comprising an array comprising a plurality of detection loci, wherein each detection locus comprises a hybridization probe that uniquely hybridizes to each unique identifier sequence.
